(19) United States
(12) Patent Application Publication
(10) Pub. No.: US 2026/0000353 A1
(43) Pub. Date: Jan. 1, 2026

(54) Novel Method for Accurate Optical Hydration Measurement Using Heartbeat-Based Analysis with Melanin Correction and Water-to-Blood Ratio Scaling

(71) Applicant: Mohsen Sharifzadeh, Cottonwood Heights, UT (US)

(72) Inventor: Mohsen Sharifzadeh, Cottonwood Heights, UT (US)

(73) Assignee: HydroTeck LLC, Cottonwood Heights, UT (US)

(21) Appl. No.: 18/760,160

(22) Filed: Jul. 1, 2024

(51) Int. Cl. A61B 5/00; A61B 5/1455

(52) U.S. Cl. CPC A61B 5/4875; A61B 5/1455; A61B 5/486; A61B 5/746

(57) ABSTRACT

Accurate hydration measurement is crucial for health. This patent presents a non-invasive method for quantifying hydration in tissues and bloodstream using heartbeat-based optical density (OD) analysis. By employing LEDs at specific wavelengths for water and blood OD measurements and correcting for melanin interference, this method calculates hydration as the ratio of water OD to blood OD. The technique offers precise, rapid, and minimally invasive hydration assessment, addressing traditional method limitations. This approach is suitable for wearable or stand-alone devices, enhancing health monitoring and personalized hydration strategies.

### BACKGROUND OF THE INVENTION

### Proposed Background for Hydration Focus

Hydration is a critical aspect of maintaining optimal health, impacting various physiological functions such as homeostasis, biochemical reactions, thermoregulation, and nutrient transport. Accurate assessment of hydration levels is essential for preventing dehydration and overhydration, both of which can lead to severe health issues. Traditional methods of hydration assessment, such as monitoring urine color and body weight changes, are often inadequate as they fail to capture the intricate distribution of water within the body.

Current techniques for measuring hydration typically involve invasive procedures, are time-consuming, and can be uncomfortable for patients. These methods often require blood samples or other bodily fluids, which can lead to patient discomfort and risk of infection. Additionally, the accuracy of these methods can be compromised by various factors, including sample contamination and degradation over time.

Non-invasive optical methods for measuring hydration have shown promise but are hindered by interference from biological pigments like melanin and hemoglobin. Melanin, which determines skin color, absorbs light across a wide spectrum, potentially skewing optical measurements. Hemoglobin, responsible for oxygen transport in blood, also absorbs light in the visible and near-infrared regions, further complicating the accuracy of hydration measurements.

To address these challenges, there is a need for a novel, non-invasive method that can accurately measure hydration levels in real-time, accounting for the interference caused by melanin and hemoglobin. Such a method would provide significant benefits in various applications, including sports, healthcare, and everyday hydration monitoring, ensuring optimal physiological functioning and overall well-being.

The present invention introduces a method that leverages heartbeat-based optical density (OD) analysis, using specific wavelengths of light to measure water and blood OD. By correcting for melanin and hemoglobin interference, this method provides a precise, rapid, and minimally invasive approach to hydration assessment. This innovative technique represents a substantial improvement over existing methods, offering a reliable solution for real-time hydration monitoring through wearable or stand-alone devices.

### DETAILED DESCRIPTION OF THE INVENTION

### 1. Overview

The invention provides a novel method for accurate hydration measurement in biological tissues and the bloodstream using heartbeat-based optical density (OD) analysis. This method employs specific wavelengths of light to measure water and blood OD and corrects for melanin interference, ensuring precise and individualized hydration assessment.

### 2. Methodology

### 1. Measurement of Water OD:

Water OD is measured in the bloodstream by leveraging heartbeat variations, focusing on the alternating current (AC) component of the optical signal.
Three LEDs are used:
   850 nm and 1085 nm for establishing a baseline.
   970 nm for measuring the peak of water absorption.

### 2. Measurement of Blood OD:

Accurate blood OD measurement is crucial for calculating hydration.
Melanin, a pigment that interferes with optical measurements, is accounted for using specific wavelengths.
Two LEDs (850 nm and 680 nm) are used to create a tangent line, extrapolated to 525 nm, serving as the baseline for blood OD measurement.
Blood OD is measured at 525 nm from the absorption spectrum relative to the baseline.

### 3. Correction for Melanin Interference:

Melanin's broad absorption spectrum is considered to ensure accurate blood OD readings.
A polynomial fitting method with three points (e.g., 700 nm, 850 nm, and 1100 nm) is used to create a baseline and correct for melanin interference.

### 4. Hydration Calculation:

Hydration is measured as the ratio of water OD to blood OD.
This ratio provides a precise indication of hydration levels, accounting for individual variations in melanin and hemoglobin.

### 3. Device Implementation

The method can be implemented in a wearable or stand-alone device designed for real-time hydration monitoring.
The device will incorporate the necessary LEDs and sensors to perform the measurements and corrections as described.
The aim is to provide a user-friendly interface that continuously monitors hydration, offering alerts and insights for optimal health management.

### 4. Applications

This technology is suitable for a wide range of applications, including sports, healthcare, and everyday hydration monitoring.
It provides significant benefits in preventing dehydration and overhydration, ensuring optimal physiological functioning.

### 5. Mathematical Algorithm for Hydration Measurement

### a) Measure Water OD at 970 nm:

$$Water OD = {OD}_{970} - ( \frac{{OD}_{850} + {OD}_{1085}}{2} )$$

### b) Generate the Tangent Line for Melanin Correction:

$$Tangent Line = a λ + b$$

where the coefficients a and b are determined by linear regression:

$$a = \frac{{OD}_{800} - {OD}_{700}}{800 - 700}$$

$$b = {OD}_{700} - a . 700$$

c) Measure Blood OD at 525 nm from the Tangent Line:
Using AC Component:

$$Blood {OD}_{AC} = {OD}_{525} - ( a \cdot 525 + b )$$

Using DC Component:

$$Blood {OD}_{DC} = {OD}_{525} - ( a \cdot 525 + b )$$

### d) Calculate Hydration Ratio:

For AC Component:

$$Hydration Ratio = \frac{Water OD}{Blood {OD}_{AC}}$$

For DC Component:

$$Hydration Ratio = \frac{Water OD}{Blood {OD}_{DC}}$$

### e) Apply Signal Processing to Measure True Hydration:

For AC Component:

$$True Hydration = Hydration {Ratio}_{AC}$$

### f) Direct Calculation for DC Component:

$$True Hydration = Hydration {Ratio}_{DC}$$

### Example Implementation

Let's start with the first part: Device Configuration. This section will describe the configuration of the device used to implement the hydration measurement method, detailing the components such as LEDs, sensors, and their arrangement.

### Device Configuration

### 1. LEDs and Sensors:

LED Configuration:
   The device employs multiple LEDs to emit light at specific wavelengths essential for measuring water and blood OD. The wavelengths include, but are not limited to, approximately 850 nm, 970 nm, and 1085 nm for water OD measurement, and approximately 850 nm, 680 nm, and 525 nm for blood OD measurement.
   These LEDs are strategically placed to ensure optimal penetration of light through the tissue and effective detection of the reflected or transmitted light by the sensors.
Sensor Configuration:
   The device includes photodetectors positioned to receive light reflected or transmitted through the tissue. These sensors are sensitive to the specific wavelengths emitted by the LEDs.
   The sensors convert the received light into electrical signals corresponding to the optical density at each wavelength.

### 2. Signal Processing:

Data Acquisition:
   The device continuously collects data from the sensors, capturing the variations in optical density as the light passes through the tissue.
   The raw data includes both the AC component, corresponding to the pulsatile changes due to the heartbeat, and the DC component, representing the steady-state absorption.
Filtering:
   The raw signals are filtered to separate the AC and DC components. High-pass filters are used to isolate the AC component, while low-pass filters are applied to extract the DC component.

### 3. Data Analysis:

Water OD Calculation:
   Water OD is calculated using the AC component of the signals at 850 nm, 970 nm, and 1085 nm. The formula used is:

$$Water OD = {OD}_{970} - ( \frac{{OD}_{850} + {OD}_{1085}}{2} )$$

Blood OD Calculation:
   Blood OD is measured at 525 nm using both the AC and DC components. A tangent line is generated using the signals at 700 nm and 800 nm to correct for melanin interference. The blood OD is then calculated as:

$$Blood {OD}_{AC} = {OD}_{525} - ( a \cdot 525 + b )$$

$$Blood {OD}_{DC} = {OD}_{525} - ( a \cdot 525 + b )$$

### 4. User Interface:

Real-Time Display:
   The device features a user interface that displays real-time hydration levels. The calculated hydration ratio is shown, and the user receives alerts if hydration levels fall outside optimal ranges.
Data Logging:
   The device logs historical hydration data, allowing users to track their hydration status over time and identify trends.

### 5. Power Supply:

Battery Configuration:
   The device is powered by a rechargeable battery, providing sufficient power for continuous monitoring. The battery life is optimized to ensure prolonged use without frequent recharging.

### Implementation Variants

### a) Medical Device Approach

### 1. Purpose and Use Case:

Designed for clinical settings, this device provides precise hydration monitoring for patients with medical conditions that require careful hydration management.
Suitable for hospitals, clinics, and home healthcare services.

### 2. Advanced Features:

Integration with Medical Records:
   The device can integrate with electronic health record (EHR) systems to provide continuous updates on a patient's hydration status.
Alerts for Healthcare Providers:
   Automatic alerts are sent to healthcare providers if a patient's hydration levels fall outside the safe range.
Detailed Analytics:
   Provides detailed analytics and trends to help healthcare providers make informed decisions about hydration management.

### 3. Sterilization and Hygiene:

Disposable Covers:
   Uses disposable covers for the sensor contact points to maintain hygiene and prevent cross-contamination between patients.
Sterilizable Components:
   Components are designed to withstand sterilization processes commonly used in medical settings.

### b) Wearable Device for Wellness

### 1. Purpose and Use Case:

Designed for general wellness and fitness monitoring, this wearable device helps users maintain optimal hydration levels during daily activities and exercise.
Suitable for athletes, fitness enthusiasts, and general health-conscious individuals.

### 2. User-Friendly Design:

Comfortable Fit:
   The device is designed to be worn comfortably on the wrist or upper arm, ensuring it does not interfere with daily activities or workouts.
Interactive App:
   Pairs with a smartphone app that provides real-time hydration data, trends, and personalized hydration tips.
Customizable Alerts:
   Users can set custom alerts to remind them to drink water based on their personal hydration needs.

### 3. Additional Features:

Fitness Tracking:
   Integrates with other fitness metrics such as heart rate, steps, and activity levels to provide comprehensive health monitoring.
Water Intake Logging:
   Allows users to log their water intake manually or through integration with smart water bottles.

### c) Patch Design for Infants and Elderlies

### 1. Purpose and Use Case:

Designed specifically for infants, elderly individuals, and others who may have difficulty using traditional wearables.
Provides non-intrusive, continuous hydration monitoring for vulnerable populations.

### 2. Design and Application:

Adhesive Patch:
   The device is a small, lightweight adhesive patch that can be comfortably worn on the skin.
Flexible and Soft Material:
   Made from flexible and soft materials that are gentle on sensitive skin, ensuring comfort and minimizing irritation.

### 3. Features:

Real-Time Monitoring:
   Continuously monitors hydration levels and sends data to a caregiver's smartphone or a dedicated monitoring device.
Safe and Hypoallergenic:
   The materials used are hypoallergenic and safe for prolonged use on all skin types.
Disposable and Replaceable:
   The patch is designed to be disposable and can be easily replaced as needed to ensure hygiene and functionality.

### 4. Caregiver Alerts:

Immediate Notifications:
   Caregivers receive immediate notifications if hydration levels are not within the optimal range.
Data Logging:
   Logs historical data to help caregivers monitor trends and make informed decisions about hydration management.

### Figure Captions:

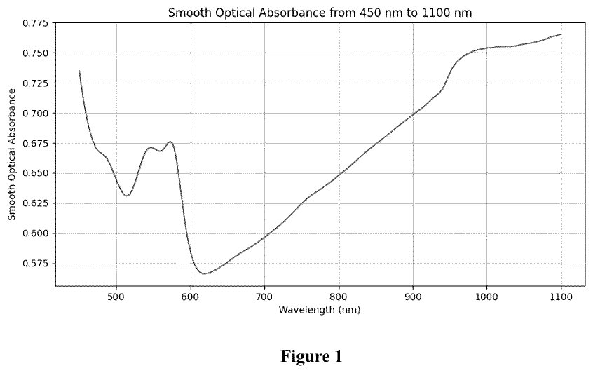

### FIG. 1

Smoothed Optical Absorbance Spectrum from 450 to 1100 nm:
This figure illustrates the smoothed optical absorbance spectrum of a sample over the wavelength range of 450 to 1100 nm.
Key absorbance peaks indicative of specific compounds are highlighted: carotenoids, oxyhemoglobin, and water.
The absorbance peaks corresponding to carotenoids are visible in the 450-500 nm range, demonstrating their characteristic light absorption properties.
Oxyhemoglobin, crucial in blood oxygen transport, shows prominent absorbance in the 540-580 nm range.
The distinct absorbance peak for water is observed at approximately 970 nm, a wavelength where water exhibits significant optical absorption.
This figure effectively demonstrates the ability of the proposed measurement method to differentiate and quantify these essential compounds within biological tissues, showcasing the method's precision and applicability across a broad spectrum.

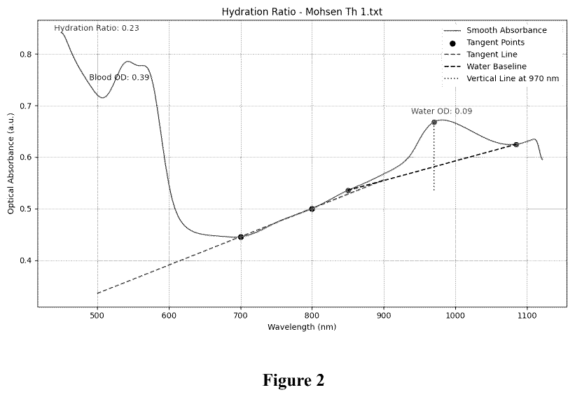

### FIG. 2

### Hydration Ratio Analysis Using Optical Absorbance Spectrum:

This figure illustrates the optical absorbance spectrum of a sample over the wavelength range of 450 to 1100 nm, highlighting the calculation of the hydration ratio.
The smooth absorbance curve represents the measured data, with tangent points used to establish baselines for both water and blood OD measurements.
The red dashed line represents the tangent line created using two predetermined wavelengths.
The black dashed line shows the baseline for water OD calculation.
The vertical dotted line at 970 nm indicates the peak of water absorption, where water OD is measured.
The water OD is calculated as 0.09, and the blood OD is calculated as 0.39.
The hydration ratio, determined as the ratio of water OD to blood OD, is shown to be 0.23.
This figure demonstrates the effectiveness of the method in accurately measuring and scaling hydration levels by correcting for melanin and hemoglobin interference.

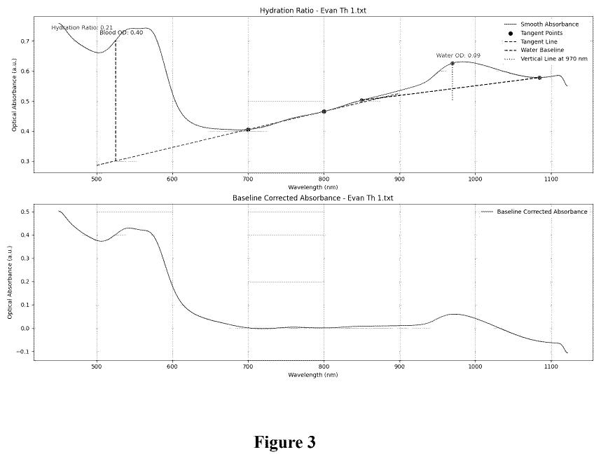

### FIG. 3

### Hydration Ratio Analysis and Baseline Corrected Absorbance Spectrum:

Upper Plot: Hydration Ratio Analysis.
   This plot illustrates the optical absorbance spectrum of a sample over the wavelength range of 450 to 1100 nm, highlighting the calculation of the hydration ratio.
   The smooth absorbance curve represents the measured data, with tangent points used to establish baselines for both water and blood OD measurements.
   The red dashed line represents the tangent line created using two predetermined wavelengths at 700 nm and 800 nm.
   The black dashed line shows the baseline for water OD calculation.
   The vertical dotted line at 970 nm indicates the peak of water absorption, where water OD is measured.
   The water OD is calculated as 0.09, and the blood OD is calculated as 0.40.
   The hydration ratio, determined as the ratio of water OD to blood OD, is shown to be 0.21.
   A dashed blue line is drawn from 525 nm to the tangent line, indicating the correction for melanin interference.
   This plot demonstrates the effectiveness of the method in accurately measuring and scaling hydration levels by correcting for melanin and hemoglobin interference.
Lower Plot: Baseline Corrected Absorbance Spectrum
   This plot shows the baseline-corrected absorbance spectrum of the sample, with corrections applied to remove the influence of melanin and other interfering factors.
   The corrected absorbance spectrum provides a clearer representation of the true absorbance characteristics of the sample, free from baseline artifacts.
   This method proves vital in ensuring accurate absorbance measurements, particularly in accounting for individual variability in melanin content.

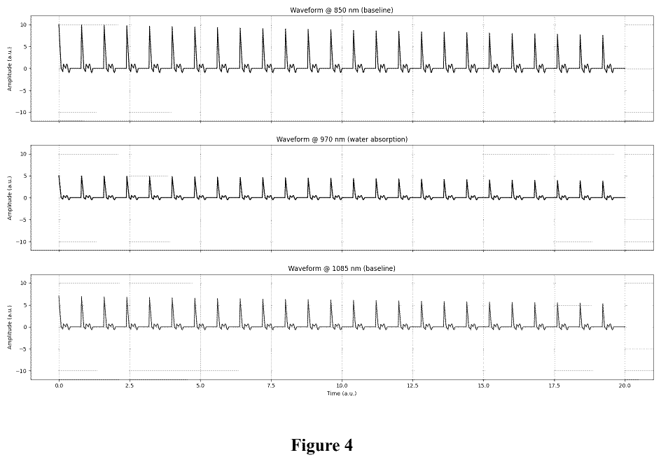

### FIG. 4

### Simulated Heartbeats at Three Different Wavelengths:

Waveform @ 850 nm (baseline):
   This subplot illustrates the simulated heartbeat waveform at 850 nm, representing a baseline measurement with the highest amplitude among the three wavelengths.
Waveform @ 970 nm (water absorption):
   This subplot shows the simulated heartbeat waveform at 970 nm, where the amplitude is the lowest due to the peak absorption of water, effectively minimizing external interferences from skin surface water or humidity.
Waveform @ 1085 nm (baseline):
   This subplot presents the simulated heartbeat waveform at 1085 nm, another baseline measurement with an amplitude higher than 970 nm but lower than 850 nm.
Common Characteristics:
   The waveforms are generated at a frequency of 1.25 Hz, corresponding to 75 heartbeats per minute.
   All waveforms share the same y-axis scale to accurately compare the amplitude differences.
   Each waveform is plotted over a 20-second duration to illustrate consistent periodicity and amplitude variation.
Hydration Measurement:
   The amplitude of the waveform at 970 nm (water absorption) is corrected by averaging the amplitudes at the baseline wavelengths (850 nm and 1085 nm) and subtracting this average from the amplitude at 970 nm. This method ensures accurate measurement of water content in the bloodstream by minimizing the influence of external factors.

This figure demonstrates the simulated heartbeats at three different wavelengths, emphasizing the differences in amplitude due to water absorption characteristics and the method used to correct the water OD measurement.

1.: Preamble: A method for accurately quantifying hydration in biological tissues and the bloodstream. Transitional Phrase: Comprising: Claim Body:
a. Measuring water optical density (OD) in the bloodstream using LEDs at specific wavelengths and leveraging the alternating current (AC) component of the optical signal corresponding to the heartbeat;
b. Establishing a baseline for water OD using at least two predetermined wavelengths, including but not limited to approximately 850 nm and 1085 nm;
c. Generating a tangent line for melanin correction using specific wavelengths, including but not limited to approximately 700 nm and 800 nm;
d. Measuring blood OD at a target wavelength, including but not limited to approximately 525 nm, from the tangent line using either the AC or DC component of the optical signal;
e. Correcting the measured blood OD for melanin interference by subtracting the value of the tangent line at the target wavelength;
f. Determining hydration as the ratio of water OD (measured using the AC component) to corrected blood OD (measured using either the AC or DC component).

2. The method of claim 1, wherein the predetermined wavelengths for measuring water OD include but are not limited to approximately 850 nm, 970 nm, and 1085 nm.

3. The method of claim 1, wherein the correction for melanin interference involves using a polynomial fitting method with at least three points to establish the baseline.

4. The method of claim 1, wherein the baseline for blood OD is created using two predetermined wavelengths, including but not limited to approximately 700 nm and 800 nm, to form a tangent line extrapolated to the target wavelength.

5. The method of claim 1, wherein the target wavelength for measuring apparent blood OD is selected based on the absorption characteristics of blood, including but not limited to approximately 525 nm.

6. The method of claim 1, wherein the LEDs used for measuring OD are configured to emit light at various wavelengths to enhance accuracy.

7. The method of claim 1, wherein the AC component of the optical signal is analyzed to account for variations due to the heartbeat for water OD measurement.

8. The method of claim 1, wherein the AC or DC component of the optical signal is analyzed to provide a stable measure of blood OD.

9. The method of claim 1, wherein the ratio of water OD (using the AC component) to corrected blood OD (using either the AC or DC component) is used to provide real-time hydration levels and insights for optimal hydration management.

10. The method of claim 1 further comprising the step of providing user alerts and recommendations based on the calculated hydration levels.

11. The method of claim 1, wherein the water OD measurement includes compensating for ambient light variations.

12. The method of claim 1 further comprising calibrating the LEDs to ensure consistent light output and accurate OD measurements.

13. The method of claim 1, wherein the polynomial fitting method for melanin correction includes dynamically adjusting based on user-specific data.

14. The method of claim 1 further comprising storing historical hydration data for trend analysis and long-term health monitoring.

15. The method of claim 1, wherein the device provides personalized hydration recommendations based on user activity levels and environmental conditions.
